(19) United States
(12) Patent Application Publication
(10) Pub. No.: US 2012/0004714 A1
(43) Pub. Date: Jan. 5, 2012

(54) LEAD HAVING COIL ELECTRODE WITH PREFERENTIAL BENDING REGION

(76) Inventors: Ryan Kleve, Maple Grove, MN (US); G. Shantanu Reddy, Minneapolis, MN (US); Bryan A. Clark, Forest Lake, MN (US)

(21) Appl. No.: 13/151,811

(22) Filed: Jun. 2, 2011

Related U.S. Application Data
(60) Provisional application No. 61/360,171, filed on Jun. 30, 2010.

(51) Int. Cl. A61N 1/05

(52) U.S. Cl. 607/116

(57) ABSTRACT

An implantable lead may have a distal shocking coil that is configured to include a predetermined buckle region in order to limit potential cardiac damage that might otherwise occur if the implantable lead is too stiff. An implantable lead may have a proximal shocking coil that is configured to have a flexibility that more closely matches the flexibility of the lead body on either side of the proximal shocking coil.

### CROSS-REFERENCE TO RELATED APPLICATION

This application claims the benefit under 35 U.S.C. §119(e) to U.S. Provisional Patent Application No. 61/360,171, filed on Jun. 30, 2010, entitled “LEAD HAVING COIL ELECTRODE WITH PREFERENTIAL BENDING REGION,” which is incorporated herein by reference in its entirety.

### TECHNICAL FIELD

The present invention relates to implantable medical devices and relates more particularly to leads for cardiac rhythm management (CRM) systems.

### BACKGROUND

Various types of medical electrical leads for use in cardiac rhythm management (CRM) and neurostimulation systems are known. For CRM systems, such leads are typically extended intravascularly to an implantation location within or on a patient's heart, and thereafter coupled to a pulse generator or other implantable device for sensing cardiac electrical activity, delivering therapeutic stimuli, and the like. The leads frequently include features to facilitate securing the lead to heart tissue to maintain the lead at its desired implantation site.

### SUMMARY

Example 1 is an implantable lead assembly that includes a flexible lead body extending between a proximal region including a proximal end and a distal region including a distal end. A distal shocking coil is disposed about the flexible lead body, the distal shocking coil located within the distal region and being configured to enable the flexible lead body to preferentially buckle within a predetermined buckle region within the shocking coil. A connector assembly is secured to the proximal end for coupling the lead to an implantable medical device, the connector assembly including a terminal pin rotatable relative to the lead body, a coil conductor disposed longitudinally within the lead body, the coil conductor rotatable relative to the lead body and coupled to the terminal pin, an electrode base rotatably disposed within the lead body, the electrode base having a proximal end and a distal end, the proximal end connected to the coil conductor, and a helical electrode fixedly secured to the electrode base, the electrode base rotatably engaged with the terminal pin via the coil conductor such that rotation of the terminal pin causes the electrode base to rotate.

In Example 2, the implantable lead of Example 1 in which the distal shocking coil includes an expanded polytetrafluoroethylene coating disposed thereon.

In Example 3, the implantable lead of Example 1 or 2 in which the distal shocking coil has a non-uniform coil pitch.

In Example 4, the implantable lead of any of Examples 1-3 in which the distal shocking coil has a non-uniform filar angle.

In Example 5, the implantable lead of any of Examples 1-4 in which the distal shocking coil includes a first coil and a second coil spaced apart from the first coil, the first and second coils electrically connected via a cable conductor extending through the flexible lead body.

In Example 6, the implantable lead of any of Examples 1-4 in which the distal shocking coil includes a multifilar coil having a reduced number of filars within the predetermined buckle region.

In Example 7, the implantable lead of Example 6 in which the distal shocking coil has two filars proximate the predetermined buckle region and three filars on either side of the predetermined buckle region.

In Example 8, the implantable lead of any of Examples 1-7, further including a proximal shocking coil disposed about the flexible lead body within the proximal region of the lead body, the proximal shocking coil being configured to have a flexibility that is substantially equivalent to that of the flexible lead body on either side of the proximal shocking coil.

In Example 9, the implantable lead of Example 8 in which the proximal shocking coil has a spaced-apart coil pitch.

Example 10 is an implantable lead that includes a flexible lead body extending between a proximal region including a proximal end and a distal region including a distal end, a coil electrode disposed about the flexible lead body, the coil electrode configured to enable the flexible lead body to preferentially buckle within a predetermined region within the coil electrode, a connector assembly secured to the proximal end for coupling the lead to an implantable medical device, and one or more electrical conductors extending through the flexible lead body, the one or more electrical connectors providing electrical communication between the connector assembly and each of the coil electrodes.

In Example 11, the implantable lead of Example 10 in which the lead is an active fixation lead.

In Example 12, the implantable lead of Example 10 in which the lead is a passive fixation lead.

In Example 13, the implantable lead of any of Examples 10-12 in which the coil electrode has a non-uniform coil pitch or a non-uniform filar angle.

In Example 14, the implantable lead of any of Examples 10-13 in which the coil electrode includes a multifilar coil having a reduced number of filars within the predetermined buckle region.

Example 15 is an implantable lead assembly that includes a flexible lead body extending between a proximal region including a proximal end and a distal region including a distal end, a lumen extending from the proximal end to the distal end. A distal shocking coil is disposed about the flexible lead body and is located within the distal region and configured to enable the flexible lead body to preferentially buckle within a predetermined lead buckle region. A connector assembly is secured to the proximal end for coupling the lead to an implantable medical device. One or more electrical conductors extend through the flexible lead body and provide electrical communication between the connector assembly and each of the shocking coils. A stylet is disposable within the lumen, the stylet including a stylet buckle region corresponding to the predetermined lead buckle region within the distal shocking coil when the stylet is extended through the predetermined lead buckle region.

In Example 16, the implantable lead assembly of Example 15 in which the stylet buckle region includes a reduced-diameter portion of the stylet.

In Example 17, the implantable lead assembly of Example 16 in which the stylet includes a distal taper and the reduced-diameter portion of the stylet is disposed proximal of the distal taper.

In Example 18, the implantable lead of any of Examples 15-17 in which the distal shocking coil has a non-uniform coil pitch or a non-uniform filar angle.

In Example 19, the implantable lead of any of Examples 15-18 in which the distal shocking coil includes a multifilar coil having a reduced number of filars within the predetermined buckle region.

In Example 20, the implantable lead of any of Examples 15-19, further including a proximal shocking coil disposed about the flexible lead body within the proximal region thereof, the proximal shocking coil having a non-uniform pitch to provide a flexibility substantially equivalent to a lead body flexibility on either side of the proximal shocking coil.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a combined cutaway and perspective view of an implantable medical device and lead in accordance with an embodiment of the present invention.

FIG. 2 is a side elevation view of the lead of FIG. 1.

FIG. 3 is a partial cross-sectional view of the lead of FIG. 1.

FIG. 4 is a cross-sectional view of a shocking coil in accordance with an embodiment of the present invention.

FIG. 5 is a cross-sectional view of a shocking coil in accordance with an embodiment of the present invention.

FIG. 6 is a cross-sectional view of a shocking coil in accordance with an embodiment of the present invention.

FIG. 7 is a cross-sectional view of a shocking coil in accordance with an embodiment of the present invention.

FIG. 8 is a cross-sectional view of a shocking coil in accordance with an embodiment of the present invention.

FIG. 9 is a partial side elevation view of a lead and a stylet in accordance with an embodiment of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

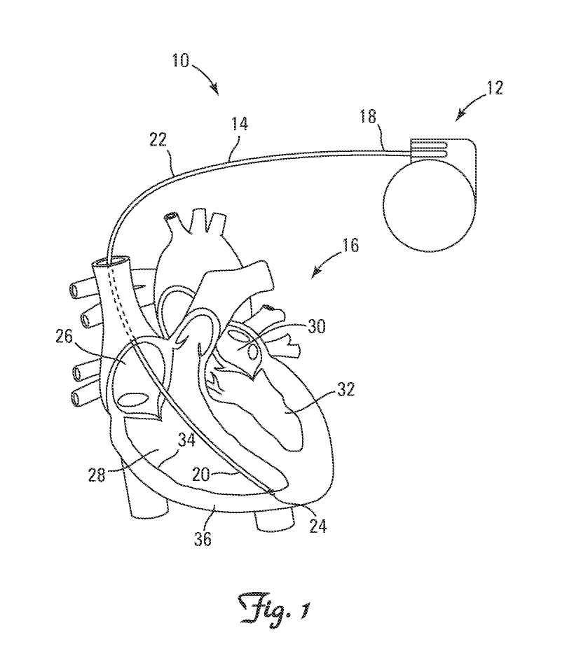

FIG. 1 is a perspective view of an implantable cardiac rhythm management (CRM) system 10. The CRM system 10 includes a pulse generator 12 and a cardiac lead 14. The lead 14 operates to convey electrical signals between the heart 16 and the pulse generator 12. The lead 14 has a proximal region 18 and a distal region 20. The lead 14 includes a lead body 22 extending from the proximal region 18 to the distal region 20. The proximal region 18 is coupled to the pulse generator 12 and the distal region 20 is coupled to the heart 16. In some embodiments, the lead 14 is an active fixation lead and the distal region 20 may, as illustrated, include an extendable/retractable fixation helix 24 which as will be discussed in greater detail below locates and/or secures the distal region 20 within the heart 16. In some embodiments, the lead 14 may be a passive fixation lead.

The pulse generator 12 is typically implanted subcutaneously within an implantation location or pocket in the patient's chest or abdomen. The pulse generator 12 may be any implantable medical device known in the art or later developed, for delivering an electrical therapeutic stimulus to the patient. In various embodiments, the pulse generator 12 is an implantable cardioverter/defibrillator (ICD), a cardiac resynchronization (CRT) device configured for bi-ventricular pacing, and/or includes combinations of pacing, CRT, and defibrillation capabilities.

The lead body 22 can be made from any flexible, biocompatible materials suitable for lead construction. In various embodiments, the lead body 22 is made from a flexible, electrically insulative material. In one embodiment, the lead body 22 is made from silicone rubber. In another embodiment, the lead body 22 is made from polyurethane. In various embodiments, respective segments of the lead body 22 are made from different materials, so as to tailor the lead body characteristics to its intended clinical and operating environments. In various embodiments, the proximal and distal ends of the lead body 22 are made from different materials selected to provide desired functionalities.

As is known in the art, the heart 16 includes a right atrium 26, a right ventricle 28, a left atrium 30 and a left ventricle 32. It can be seen that the heart 16 includes an endothelial inner lining or endocardium 34 covering the myocardium 36. In some embodiments, as illustrated, the fixation helix 24, located at the distal region 20 of the lead, penetrates through the endocardium 34 and is imbedded within the myocardium 36. In one embodiment, the CRM system 10 includes a plurality of leads 14. For example, it may include a first lead 14 adapted to convey electrical signals between the pulse generator 12 and the right ventricle 28 and a second lead (not shown) adapted to convey electrical signals between the pulse generator 12 and the right atrium 26.

In the illustrated embodiment shown in FIG. 1, the fixation helix 24 penetrates the endocardium 34 of the right ventricle 28 and is embedded in the myocardium 36 of the heart 16. In some embodiments, the fixation helix 24 is electrically active and thus can be used to sense the electrical activity of the heart 16 and/or to apply a stimulating pulse to the right ventricle 28. In other embodiments, the fixation helix 24 is not electrically active. Rather, in some embodiments, other components of the lead 14 are electrically active.

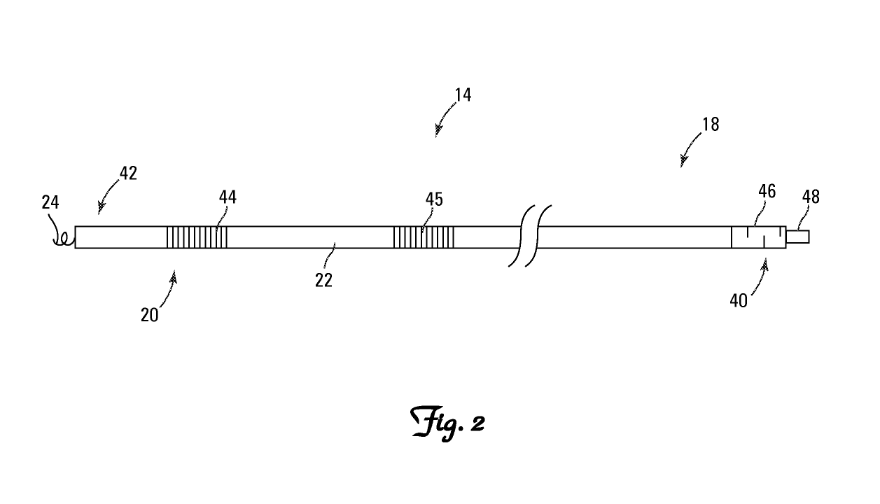

FIG. 2 is an isometric illustration of the lead 14 according to one embodiment. A connector assembly 40 is disposed at or near the proximal region 18 of the lead 14 while a distal assembly 42 is disposed at or near the distal region 20 of the lead 14. Depending on the functional requirements of the CRM system 10 (see FIG. 1) and the therapeutic needs of a patient, the distal region 20 may include one or more electrodes. In the illustrated embodiment, the distal region 20 includes a distal shocking coil 44 and a proximal shocking coil 45 that can function as shocking electrodes for providing a defibrillation shock to the heart 16.

In various embodiments, the lead 14 may include only a single shocking coil. In various other embodiments, the lead 14 includes one or more ring electrodes (not shown) along the lead body 22 in lieu of or in addition to the shocking coils 44, 45. When present, the ring electrodes operate as relatively low voltage pace/sense electrodes. In short, a wide range of electrode combinations may be incorporated into the lead 14 within the scope of the various embodiments of the present invention. In some embodiments, the distal shocking coil 44 and/or the proximal shocking coil 45 may be configured to preferentially bend or buckle in a predetermined buckle region in order to reduce the lead's column strength.

The connector assembly 40 includes a connector 46 and a terminal pin 48. The connector 46 is configured to be coupled to the lead body 22 and is configured to mechanically and electrically couple the lead 14 to a header on the pulse generator 12 (see FIG. 1). In various embodiments, the terminal pin 48 extends proximally from the connector 46 and in some embodiments is coupled to a conductor member (not visible in this view) that extends longitudinally within the lead body 22 and which is rotatable relative to the lead body 22 such that rotating the terminal pin 48 (relative to the lead body 22) causes the conductor member to rotate within the lead body 22 as well.

In some embodiments, the terminal pin 48 includes an aperture extending therethrough, and the conductor member defines a longitudinal lumen in communication with the aperture. When present, the aperture and/or conductor lumen are configured to accommodate a guide wire or an insertion stylet for delivery of the lead 14.

The distal assembly 42 includes a housing, within which the fixation helix 24 is at least partially disposed. In some embodiments, the housing includes or accommodates a mechanism that enables the fixation helix 24 to move distally and proximally relative to the housing. In some embodiments, as the lead 14 may be a passive fixation lead and thus may not include the fixation helix 24.

In some embodiments, the housing may accommodate or include structure that limits distal travel of the fixation helix 24 (relative to the housing). As noted above, the fixation helix 24 operates as an anchoring means for anchoring the distal region 20 of the lead 14 within the heart 16. In some embodiments, the fixation helix 24 is electrically active, and is also used as a pace/sense electrode. In some embodiments, the fixation helix 24 is made of an electrically conductive material such as Elgiloy, MP35N, tungsten, tantalum, iridium, platinum, titanium, palladium, stainless steel, as well as alloys of any of these materials. In some embodiments, the fixation helix 24 is made of a non-electrically conductive material such as PES (polyethersulfone), polyurethane-based thermoplastics, ceramics, polypropylene and PEEK (polyetheretherketone).

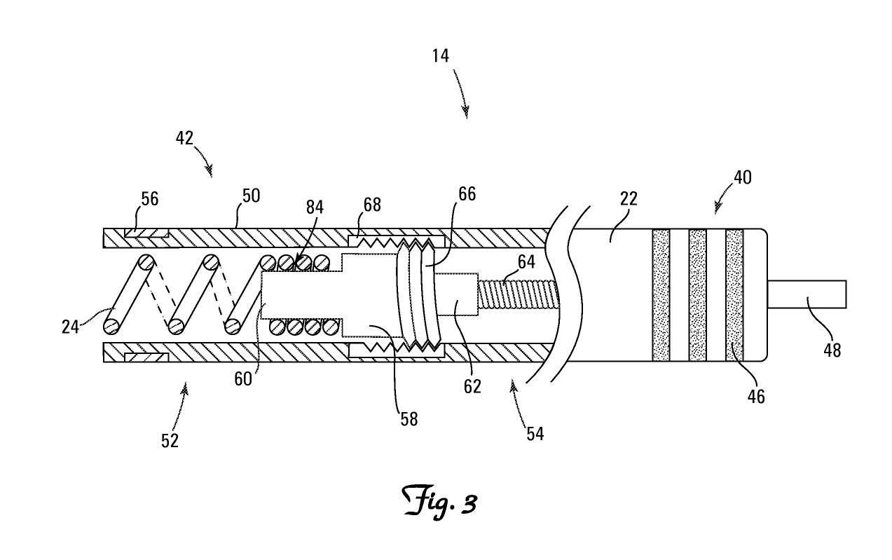

FIG. 3 illustrates an embodiment of a lead including a distal assembly in accordance with one embodiment of the present invention. In FIG. 3, the fixation helix 24 is illustrated in a retracted position. In the illustrated embodiment, the fixation helix 24 is electrically active so as to be operable as a pace/sense electrode.

As shown in FIG. 3, the distal assembly 42 includes a distal region 52 and a proximal region 54. The distal assembly 42 is, in general, relatively rigid or semi-rigid. In some embodiments, the distal assembly 42 is made of an electrically conductive material such as Elgiloy, MP35N, tungsten, tantalum, iridium, platinum, titanium, palladium, stainless steel, as well as alloys of any of these materials. In some embodiments, the distal assembly 42 is made of a non-electrically conductive material such as PES, polyurethane-based thermoplastics, ceramics, polypropylene and PEEK.

In the illustrated embodiment, a drug eluting collar 56 is disposed about an exterior of the distal assembly 42 within the distal region 52. In various embodiments, the drug eluting collar 56 is configured to provide a time-released dosage of a steroid or other anti-inflammatory agent to the tissue to be stimulated, e.g., the heart tissue in which the electrically active fixation helix 24 is implanted. While not illustrated, in some embodiments the distal assembly 42 may include a radiopaque element disposed under the drug eluting collar 56.

As shown, the distal assembly 42 includes an electrode base 58. In some embodiments, the electrode base 58 is made of an electrically conductive material such as Elgiloy, MP35N, tungsten, tantalum, iridium, platinum, titanium, palladium, stainless steel, as well as alloys of any of these materials. In some embodiments, the electrode base 58 is made of a non-electrically conductive material such as PES (polyethersulfone), polyurethane-based thermoplastics, ceramics, polypropylene and PEEK (polyetheretherketone).

In some embodiments, the electrode base 58 is configured to move longitudinally and/or rotationally with respect to the distal assembly 42. As illustrated, the electrode base 58 includes a distal portion 60 and a proximal portion 62. As shown, the fixation helix 24 is connected to the distal portion 60 of the electrode base 58. In some embodiments, as illustrated, the distal portion 60 may have a relatively smaller diameter that is configured to accommodate the fixation helix 24. In some embodiments, the proximal portion 62 of the electrode base 58 may be configured to accommodate a seal (not illustrated).

A conductor coil 64 is secured to the proximal portion 62 of the electrode base 58, and extends proximally through the lead body 22 to the connector assembly 40. In some embodiments, the conductor coil 64 is welded or soldered to the proximal portion 62 of the electrode base 58. In some embodiments, the conductor coil 64 includes or is otherwise formed from a metallic coil.

In the connector assembly 40, the conductor coil 64 is coupled to the terminal pin 48 such that rotation of the terminal pin 48 causes the conductor coil 64 to rotate. As the conductor coil 64 rotates, the electrode base 58 and the fixation helix 24 will also rotate. In some embodiments, the fixation helix 24 is rotated via a stylet that is inserted through an aperture that may be formed within the terminal pin 48 (FIG. 2).

In some embodiments, as illustrated, the electrode base 58 includes a threaded portion 66 that interacts with a corresponding threaded portion 68 secured within the distal assembly 42. It will be appreciated that relative rotation between the threaded portion 66 and the threaded portion 68 will cause the electrode base 58 to translate or move axially relative to the distal assembly 42. For example, the threaded portion 66 and the threaded portion 68 may be configured such that rotating the terminal pin 48 in a clockwise fashion will cause the electrode base 58 and the fixation helix 24 to move outwards towards an extended position while counter clockwise rotation of the terminal pin 48 may retract the electrode base 58 and the fixation helix 24.

The particular arrangement illustrated in FIG. 3 facilitating extension and retraction of the fixation helix 24 is exemplary only. In other words, any arrangement, whether now known or later developed, for providing the extendable/retractable functionality of the fixation helix 24 can be utilized in connection with the various embodiments of the present invention. In one embodiment, the lead 14 includes structures such as those described and illustrated in co-pending and commonly assigned U.S. Provisional Patent Application 61/181,954, the disclosure of which is incorporated by reference herein in its entirety. In other embodiments, a different arrangement for extending and retracting the fixation helix 24 is utilized.

In some embodiments, the distal shocking coil 44 and/or the proximal shocking coil 45 may include a coating that inhibits tissue ingrowth yet permits electrical charges to pass from the shocking coil 44, 45 to the surrounding heart tissue. In some embodiments, the coating is a porous polymer coating that inhibits tissue ingrowth yet permits ions to pass through. An exemplary coating is an expanded polytetrafluoroethylene. In some embodiments, inclusion of the coating may stiffen the coil by limiting relative movement between adjacent filars. In some embodiments, the shocking coil may be modified or configured to bend or buckle within a predetermined buckle region.

In some embodiments, the distal shocking coil 44 may be configured to preferentially bend or buckle within a predetermined buckle region. In some embodiments, the predetermined buckle region may correspond to a point within the distal shocking coil 44. In some embodiments, the proximal shocking coil 45 may be configured to have a flexibility that more closely matches a flexibility of the lead body 22 on either side of the proximal shocking coil 45. FIGS. 4-8 provide illustrative examples of how the distal shocking coil 44 and/or the proximal shocking coil 45 may be configured to provide desired bending and/or flexibility characteristics.

FIG. 4 is a partial cross-section view of a coil 70 that may be used as the distal shocking coil 44 and/or the proximal shocking coil 45. While the coil 70 is illustrated as including a single filar 72, the coil 70 may, in some embodiments, be formed as a multi-filar coil. In some embodiments, the coil 70 may be defined at least in part based upon its pitch. Pitch refers to the spacing between adjacent filar turnings. For example, a tight pitch may be defined as referring to a coil or a portion of a coil in which adjacent filar turnings are touching or slightly spaced apart. In contrast, an expanded pitch may be defined as referring to a coil or a portion of a coil in which adjacent filar turnings are spaced farther apart.

The coil 70 has a distal region 74 having a tight coil pitch, a proximal region 76 having a tight coil pitch and a predetermined buckle region 78 in which the coil 70 has an expanded coil pitch. As an illustrative but non-limiting example, assume that coil 70 is formed of three adjacent filars 72, each having a diameter of 0.008 inches. If the filars 72 are in close contact, the coil 70 would be considered as having a pitch of 0.024 inches (3 times 0.008 inches). In this example, the distal region 74 and the proximal region 76 may each have a pitch that is in the range of about 0.024 inches to about 0.028 inches while the predetermined buckle region 78 may have a pitch that is in the range of about 0.028 inches to about 0.040 inches. It should be noted that these ranges are merely illustrative and may vary, depending upon the number of filars 72 and the diameters of the filars 72.

FIG. 5 is a partial cross-section view of a coil 80 that is similar to the coil 70, but has a less drastic pitch change. The coil 80 may be used as the distal shocking coil 44 and/or the proximal shocking coil 45. In some embodiments, as illustrated, the coil 80 is formed from a single filar 72. In other embodiments, the coil 80 may be formed as a multi-filar coil. The coil 80 has a distal region 82 having a tight coil pitch, a proximal region 84 having a tight coil pitch and a predetermined buckle region 86 in which the coil 80 has an expanded coil pitch. In the illustrated embodiment, the distal region 82 and the proximal region 84 both have a tight coil pitch in which adjacent filar turnings are in contact. In some embodiments, the distal region 82 and/or the proximal region 84 may have a coil pitch in which adjacent filar turnings are slightly spaced apart, but not as spaced apart as the adjacent filar turnings within the predetermined buckle region 86.

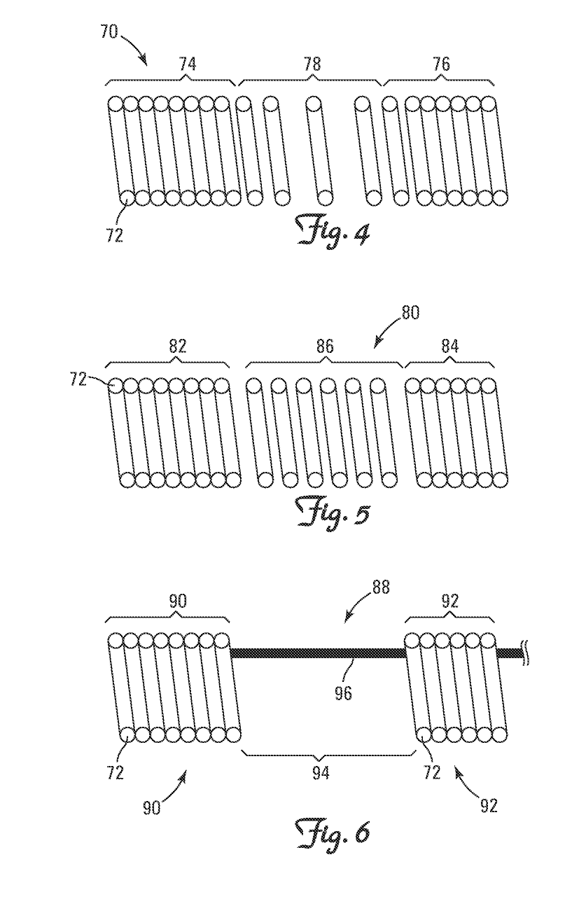

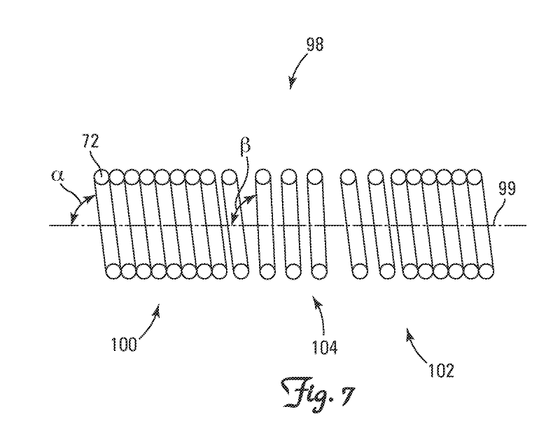

FIG. 6 is a partial cross-section of a coil assembly 88 that may be used as the distal shocking coil 44 and/or the proximal shocking coil 45. The coil assembly 88 includes a distal coil 90, a proximal coil 92 and an intervening predetermined buckle region 94 in which there is no coil filars extending therethrough. The distal coil 90 and the proximal coil 92 are each, as illustrated, formed of a single filar 72, although in some embodiments the distal coil 90 and/or the proximal coil 92 may be multi-filar coils.

The distal coil 90 and the proximal coil 92 are electrically connected together by being secured to a cable conductor 96 that extends between the distal coil 90 and the proximal coil 92. In some embodiments, the cable conductor 96 extends through the lead body 22 back to the connector assembly 40. In some embodiments, a fitting (not illustrated) may be welded to at least one of the distal coil 90 and the proximal coil 92, and the cable conductor 96 can be crimped into the fitting to provide electrical contact as well as a secure attachment between the coil 90 or 92 and the cable conductor 96.

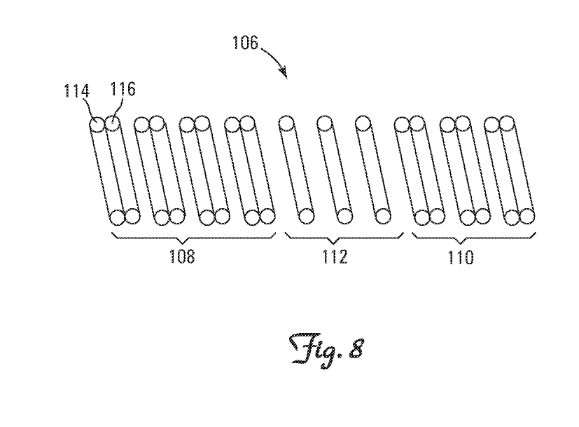

FIG. 7 is a partial cross-section of a coil 98 that may be used as the distal shocking coil 44 and/or the proximal shocking coil 45. The coil 98 is illustrated as being formed from a single filar 72, although in some embodiments the coil 98 may be configured as a multi-filar coil. The coil 98 includes a distal region 100 having a first filar angle alpha measured with respect to a longitudinal axis 99, a proximal region 102 having the same filar angle alpha and an intermediate predetermined buckle region 104 having a second filar angle beta.

In some embodiments, beta may be an acute angle that is greater than alpha. In some embodiments, the relative stiffness of the predetermined buckle region 104 may be altered by increasing or decreasing the filar angle beta within the predetermined buckle region 104. In some embodiments, the coil pitch as well as the filar angle may be altered in the predetermined buckle region 104.

In some embodiments, the filar angle alpha may be in the range of about 45 degrees to about 80 degrees while the filar angle beta may be in the range of about 70 degrees to about 90 degrees. In some embodiments, these values may vary, depending on various factors such as the coil size and filar diameter. In some embodiments, the relative values for alpha and beta may not be as important as the difference between alpha and beta.

FIG. 8 is a partial cross-section of a coil 106 that may be used as the distal shocking coil 44 and/or the proximal shocking coil 45. The coil 106 includes a distal region 108, a proximal region 110 and a predetermined buckle region 112 disposed between the distal region 108 and the proximal region 110. In this embodiment, the distal region 108 and the proximal region 110 include a two-filar configuration having a first filar 114 and a second filar 116 while the predetermined buckle region 112 only includes the first filar 114. In some embodiments, the second filar 116 may be removed from the predetermined buckle region 112 while in other embodiments the predetermined buckle region 112 may be formed with only a single filar.

In some embodiments, other filar counts may also be used. For example, the distal region 108 and the proximal region 110 may include two, three, four or more filars while the predetermined buckle region 112 may include one or more fewer filars than used in the distal region 108 and the proximal region 110. In some embodiments, the entire coil 106 may have a constant filar count of one, two, three or more filars, but the filar(s) may have a non-constant diameter. For example, in some embodiments, the filar(s) may have a relatively larger diameter within the distal region 108 and the proximal region 110 and a relative smaller diameter within the predetermined buckle region 112.

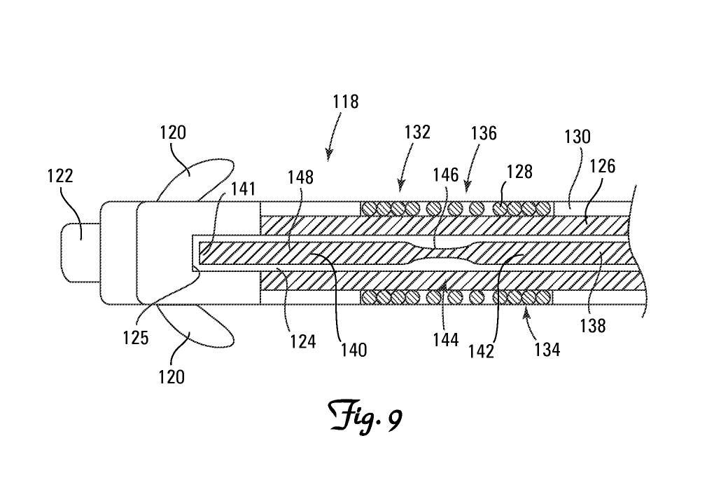

In some embodiments, the lead 14 may be deployed using a stylet to stiffen the lead 14 and/or provide better steerability to the lead 14. In some embodiments, and as illustrated in FIG. 9, a stylet may be configured to have a stylet buckle location that corresponds to the predetermined buckle location of the distal shocking coil 44. While FIG. 9 illustrates a passive fixation lead, it will be appreciated that a similar stylet may be used in combination with an active fixation lead such as that described above.

FIG. 9 is a partial cross-section of a distal region of a passive fixation lead 118. The passive fixation lead 118 includes a plurality of fixation wings 120 and a distal electrode 122. A lumen 124 extends through the lead 118 and includes a terminus 125 that is configured to accommodate a distal end of a stylet. The lumen 124 is defined at least in part by a first polymeric layer 126. A coil 128 that as discussed above may be a single filar coil or a multi-filar coil is wound around the first polymeric layer 126. A second polymeric layer 130 extends around the first polymeric layer 126 and provides the lead 118 with a constant outer diameter. The coil 128 includes a distal region 132, a proximal region 134 and an intervening predetermined buckle region 136 in which the coil 128 has an increased pitch (distance between adjacent turnings).

A stylet 138 extends through the lumen 124. The stylet includes a distal region 140 including a distal end 141, a proximal region 142 and an intervening stylet buckle region 144. In some embodiments, as illustrated, the intervening stylet buckle location 144 includes a narrowed portion 146 within a stylet shaft 148. The narrowed portion 146 provides a reduced stiffness region that corresponds to the predetermined buckle region 136 of the coil 128.

In some embodiments, the stylet buckle region 144 may instead have an increased diameter or otherwise be configured to be stiffer than the rest of the stylet 138. In some embodiments, a relatively stiffer stylet buckle region 144 may counteract the flexibility of the predetermined buckle region 136 of the coil 128 in order to improve maneuverability during lead implantation.

The coil configurations described and illustrated herein have been described as shocking coils. In some embodiments, these coil configurations may be used as other types of coil electrodes in a variety of different lead types including brady leads, pacing leads and the like.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An implantable lead assembly comprising:
a flexible lead body extending between a proximal region including a proximal end and a distal region including a distal end;
a distal shocking coil disposed about the flexible lead body, the distal shocking coil located within the distal region and configured to enable the flexible lead body to preferentially buckle within a predetermined buckle region within the shocking coil;
a connector assembly secured to the proximal end for coupling the lead to an implantable medical device, the connector assembly including a terminal pin rotatable relative to the lead body;
a coil conductor disposed longitudinally within the lead body, the coil conductor rotatable relative to the lead body and coupled to the terminal pin;
an electrode base rotatably disposed within the lead body, the electrode base having a proximal end and a distal end, the proximal end connected to the coil conductor; and
a helical electrode fixedly secured to the electrode base, the electrode base rotatably engaged with the terminal pin via the coil conductor such that rotation of the terminal pin causes the electrode base to rotate.

2. The implantable lead of claim 1, wherein the distal shocking coil includes an expanded polytetrafluoroethylene coating disposed thereon.

3. The implantable lead of claim 1, wherein the distal shocking coil has a non-uniform coil pitch.

4. The implantable lead of claim 1, wherein the distal shocking coil has a non-uniform filar angle.

5. The implantable lead of claim 1, wherein the distal shocking coil comprises a first coil and a second coil spaced apart from the first coil, the first and second coils electrically connected via a cable conductor extending through the flexible lead body.

6. The implantable lead of claim 1, wherein the distal shocking coil comprises a multifilar coil having a reduced number of filars within the predetermined buckle region.

7. The implantable lead of claim 6, wherein the distal shocking coil has two filars proximate the predetermined buckle region and three filars on either side of the predetermined buckle region.

8. The implantable lead of claim 1, further comprising a proximal shocking coil disposed about the flexible lead body within the proximal region of the lead body, the proximal shocking coil being configured to have a flexibility that is substantially equivalent to that of the flexible lead body on either side of the proximal shocking coil.

9. The implantable lead of claim 8, wherein the proximal shocking coil has a spaced-apart coil pitch.

10. An implantable lead comprising:
a flexible lead body extending between a proximal region including a proximal end and a distal region including a distal end;
a coil electrode disposed about the flexible lead body, the coil electrode configured to enable the flexible lead body to preferentially buckle within a predetermined region within the coil electrode;
a connector assembly secured to the proximal end for coupling the lead to an implantable medical device; and
one or more electrical conductors extending through the flexible lead body, the one or more electrical connectors providing electrical communication between the connector assembly and each of the coil electrodes.

11. The implantable lead of claim 10, wherein the lead is an active fixation lead.

12. The implantable lead of claim 10, wherein the lead is a passive fixation lead.

13. The implantable lead of claim 10, wherein the coil electrode has a non-uniform coil pitch or a non-uniform filar angle.

14. The implantable lead of claim 10, wherein the coil electrode comprises a multifilar coil having a reduced number of filars within the predetermined buckle region.

15. An implantable lead assembly comprising:
a flexible lead body extending between a proximal region including a proximal end and a distal region including a distal end, a lumen extending from the proximal end to the distal end;
a distal shocking coil disposed about the flexible lead body, the distal shocking coil located within the distal region and configured to enable the flexible lead body to preferentially buckle within a predetermined lead buckle region;
a connector assembly secured to the proximal end for coupling the lead to an implantable medical device;
one more electrical conductors extending through the flexible lead body, the one or more electrical connectors providing electrical communication between the connector assembly and each of the shocking coils; and
a stylet disposable within the lumen, the stylet including a stylet buckle region corresponding to the predetermined lead buckle region within the distal shocking coil when the stylet is extended through the predetermined lead buckle region.

16. The implantable lead assembly of claim 15, wherein the stylet buckle region comprises a reduced-diameter portion of the stylet.

17. The implantable lead assembly of claim 16, wherein the stylet comprises a distal taper and the reduced-diameter portion of the stylet is disposed proximal of the distal taper.

18. The implantable lead of claim 16, wherein the distal shocking coil has a non-uniform coil pitch or a non-uniform filar angle.

19. The implantable lead of claim 16, wherein the distal shocking coil comprises a multifilar coil having a reduced number of filars within the predetermined buckle region.

20. The implantable lead of claim 16, further comprising a proximal shocking coil disposed about the flexible lead body within the proximal region thereof, the proximal shocking coil having a non-uniform pitch to provide a flexibility substantially equivalent to a lead body flexibility on either side of the proximal shocking coil.
